(19) United States
(12) Plant Patent Application Publication
(10) Pub. No.: US 2016/0353630 P1
(43) Pub. Date: Dec. 1, 2016

(54) HESPEROZYGIS PLANT NAMED 'HES040302"

(50) ;

(71) Applicant: Ushio Sakazaki, Hikone (JP)

(72) Inventor: Ushio Sakazaki, Hikone (JP)

(73) Assignee: EUROAMERICAN PROPAGATORS LLC, BONSALL, CA (US)

(21) Appl. No.: 14/545,620

(22) Filed: May 29, 2015

(51) Int. Cl. A01H 5/00

(52) U.S. Cl. PLT/263.1

(57) ABSTRACT

A new and distinct Hesperozygis plant named ‘HES040302’, characterized by its compact, upright to outwardly spreading, mounding to trailing plant habit; freely branching habit; dense and bushy appearance; early and freely flowering habit; flowers that are initially orange in color and become dark pink in color with development; long flowering period; and good garden performance.

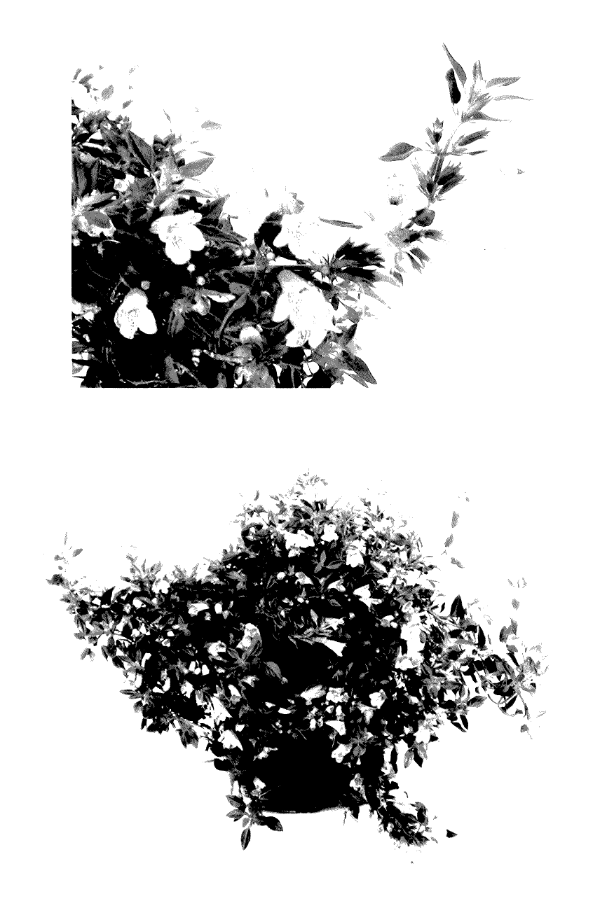

### BOTANICAL DESIGNATION

Hesperozygis hybrida×Satureja hybrida

### CULTIVAR DENOMINATION

‘HES040302’

### BACKGROUND OF THE INVENTION

The present invention relates to a new and distinct cultivar of Hesperozygis plant, botanically known as Hesperozygis hybrida×Satureja hybrida and hereinafter referred to by the name ‘HES040302’.

The new Hesperozygis plant is a product of a planned breeding program conducted by the Inventor in Higashiomi, Shiga, Japan. The objective of the breeding program is to create new uniform and freely-branching Hesperozygis plants with fragrance and attractive flowers.

The new Hesperozygis plant originated from a cross-pollination made by the Inventor on Apr. 19, 2003 in Higashiomi, Shiga, Japan of a proprietary selection of Hesperozygis hybrida identified as code number 02BR-55, not patented, as the female, or seed, parent with an unnamed proprietary selection of Satureja hybrida, not patented, as the male, or pollen, parent. The new Hesperozygis plant was discovered and selected by the Inventor as a single flowering plant within the progeny of the stated cross-pollination in a controlled environment in Higashiomi, Shiga, Japan on Sep. 20, 2004.

Asexual reproduction of the new Hesperozygis plant by vegetative terminal cuttings in a controlled environment in Higashiomi, Shiga, Japan since Sep. 25, 2007 has shown that the unique features of this new Hesperozygis plant are stable and reproduced true to type in successive generations.

### SUMMARY OF THE INVENTION

Plants of the new Hesperozygis have not been observed under all possible combinations of environmental conditions and various cultural practices. The phenotype may vary somewhat with variations in environmental conditions such as temperature and light intensity without, however, any variance in genotype.

The following traits have been repeatedly observed and are determined to be the unique characteristics of ‘HES040302’. These characteristics in combination distinguish ‘HES040302’ as a new and distinct Hesperozygis plant:
1. Compact, upright to outwardly spreading, mounding to trailing plant habit.
2. Freely branching habit; dense and bushy appearance.
3. Early and freely flowering habit.
4. Flowers that are initially orange in color and become dark pink in color with development.
5. Long flowering period.
6. Good garden performance.

The new Hesperozygis plant can be compared to plants of the female parent selection. Plants of the new Hesperozygis differ primarily from plants of the female parent selection in flower color as plants of the female parent selection have pink-colored flowers that do not change in color with development. In addition, plants of the new Hesperozygis flower for a longer period of time than plants of the female parent selection.

The new Hesperozygis plant can be compared to plants of the male parent selection. Plants of the new Hesperozygis differ primarily from plants of the male parent selection in flower color as plants of the male parent selection have orange-colored flowers that do not change in color with development. In addition, plants of the new Hesperozygis flower for a longer period of time than plants of the male parent selection.

Plants of the new Hesperozygis can be compared to plants of Saturozygis hybrid ‘Usmint2’, disclosed in U.S. Plant Pat. No. 16,790. In side-by-side comparisons plants of the new Hesperozygis differed primarily from plants of ‘Usmint2’ in flower size and color as plants of ‘Usmint2’ had longer pink-colored flowers that did not change in color with development. In addition, plants of the new Hesperozygis had lighter green-colored leaves than plants of ‘Usmint2’.

Plants of the new Hesperozygis can also be compared to plants of Hesperozygis ‘Sunminbu’, disclosed in U.S. Plant Pat. No. 12,677. In side-by-side comparisons plants of the new Hesperozygis differed primarily from plants of ‘Sunminbu’ in flower color as plants of ‘Sunminbu’ had blue-colored flowers that did not change in color with development. In addition, plants of the new Hesperozygis flowered earlier than plants of ‘Sunminbu’.

### BRIEF DESCRIPTION OF THE PHOTOGRAPHS

The accompanying colored photographs illustrate the overall appearance of the new Hesperozygis plant showing the colors as true as it is reasonably possible to obtain in colored reproductions of this type. Colors in the photographs may differ slightly from the color values cited in the detailed botanical description which accurately describe the colors of the new Hesperozygis plant. The photograph at the bottom of the sheet comprises a side perspective view of a typical flowering plant of ‘HES040302’ grown in a container. The photograph at the top of the sheet is a close-up view of a typical flowering plant of ‘HES040302’.

### DETAILED BOTANICAL DESCRIPTION

The aforementioned photographs and following observations, measurements and values describe plants grown during the winter and early spring in 16.5-cm containers in a coldframe greenhouse in Bonsall, Calif. During the production of the plants, day temperatures ranged from 10° C. to 35° C. and night temperatures ranged from 7° C. to 21° C. Plants were 24 weeks old when the photographs and description were taken. In the following description, color references are made to The Royal Horticultural Society Colour Chart, 2007 Edition, except where general terms of ordinary dictionary significance are used.
Botanical classification: Hesperozygis hybrida×Satureja hybrida ‘HES040302’.
Parentage:
   Female, or seed, parent.—Proprietary selection of Hesperozygis hybrida identified as code number 02BR-55, not patented.
   Male, or pollen, parent.—Unnamed proprietary selection of Satureja hybrida, not patented.
Propagation:
   Type.—By vegetative terminal cuttings.
   Time to initiate roots, summer.—About three weeks at temperatures ranging from 24° C. to 27° C.
   Time to initiate roots, winter.—About four weeks at temperatures ranging from 21° C. to 24° C.
   Time to produce a rooted plant, summer.—About six weeks at temperatures ranging from 24° C. to 27° C.
   Time to produce a rooted plant, winter.—About seven weeks at temperatures ranging from 18° C. to 21° C.
   Root description.—Fine, fibrous; white in color.
   Rooting habit.—Freely branching; medium density.
Plant description:
   Plant and growth habit.—Compact, upright to outwardly spreading, mounding to trailing plant habit; freely branching habit with about eight primary lateral branches each with multiple secondary branches; pinching enhances branching; dense and bushy appearance; vigorous growth habit.
   Plant height.—About 13.8 cm.
   Plant diameter.—About 38 cm by 46 cm.
Lateral branch description:
   Length.—Highly variable, about 30 cm.
   Diameter.—About 2 mm.
   Internode length.—About 2 cm.
   Strength.—Moderately strong.
   Aspect.—Initially upright then outwardly spreading to trailing.
   Texture.—Pubescent, minute.
   Color, developing.—Close to 146B.
   Color, developed.—Close to N199B.
Leaf description:
   Arrangement.—Opposite; leaves simple.
   Fragrance.—Strongly fragrant; spicy.
   Length.—About 2.2 cm.
   Width.—About 1.2 cm.
   Shape.—Elliptical.
   Apex.—Acute.
   Base.—Rounded, equilateral.
   Margin.—Mostly entire with two or three shallow serrations.
   Texture, upper and lower surfaces.—Sparsely pubescent, minute.
   Venation pattern.—Pinnate; arcuate.
   Color.—Developing leaves, upper surface: Close to N137B. Developing leaves, lower surface: Close to 146A. Fully expanded leaves, upper surface: Close to N137A; venation, close to 147B. Fully expanded leaves, lower surface: Close to 147B; venation, close to 146C.
   Petioles.—Length: About 7 mm. Diameter: About 1 mm. Texture, upper and lower surfaces: Pubescent, minute. Color, upper and lower surfaces: Close to 146C.
Flower description:
   Flower type and flowering habit.—Single salverform flowers arising from leaf axils; freely flowering habit; typically in clusters with about six per leaf axil, about twelve per node and more than 1,500 flowers developing per plant; flowers face upright to leaning outwardly.
   Fragrance.—None detected.
   Natural flowering season.—Early flowering habit, plants of the new Hesperozygis begin flowering about ten weeks after planting; plants flower continuously from spring though the summer until autumn in Southern California.
   Flower longevity.—Individual flowers last about five days on the plant; flowers not persistent.
   Flower buds.—Length: About 1.5 cm. Diameter: About 3 mm. Shape: Oblong. Texture: Pubescent, minute. Color: Close to 161A.
   Flower diameter.—About 1.2 cm.
   Flower length.—About 2.5 cm.
   Flower throat diameter.—About 6 mm by 8 mm.
   Flower tube length.—About 2.5 cm.
   Flower tube diameter, base.—About 2 mm.
   Corolla.—Arrangement: Five fused petals opening into a flared tube. Petal lobe length (from throat): About 2 mm to 4 mm. Petal lobe width: About 2 mm to 6 mm. Petal shape: Oval. Petal apex: Rounded. Petal margin: Entire. Petal texture, upper surface: Smooth, glabrous. Petal texture, lower surface: Pubescent, minute. Throat texture: Smooth, glabrous. Tube texture: Pubescent, minute. Color: Petal lobe, when opening, upper surface: Close to 163B. Petal lobe, when opening, lower surface: Close to 163B to 163C. Petal lobe, fully opened, upper surface: Close to 164C tinted with close to 179C; shifting to close to 182C to 182D; then shifting to close to 186C to 186D; and then shifting to N78C to N78D; with further development, color becomes closer to 75A to 75B. Petal lobe, fully opened, lower surface: Close to 167D and shifting to close to 77B; with further development, color becomes closer to 84C. Throat: Same as upper lobe colors with irregular splotches (nectar guides) initially close to 183C and shifting to close to 77B with development. Tube: Close to 167D and shifting to close to 77B; with further development, color becomes closer to 84C.
   Calyx.—Arrangement: Tubular calyx with five sepals fused towards the base and arranged in a single whorl. Sepal length: About 8 mm. Sepal width: About 1 mm. Sepal shape: Lanceolate. Sepal apex: Acuminate. Sepal margin: Entire. Sepal texture, upper surface: Smooth, glabrous; towards the apex, pubescent, minute. Sepal texture, lower surface: Pubescent, minute. Sepal color, upper surface: Close to 146A. Sepal color, lower surface: Close to 146B.
   Peduncles.—Length: About 3 mm to 4 mm. Diameter: About 1 mm. Angle: About 15° to 25° from stem axis. Strength: Moderately strong. Texture: Pubescent, minute. Color: Close to 146B.
   Reproductive organs.—Stamens: Quantity: Four per flower; adnate to the throat. Filament length: About 1 cm. Filament color: Close to NN155D. Anther length: About 1.5 mm. Anther shape: Lanceolate. Anther color: Close to 77D. Pollen amount: Scarce. Pollen color: Close to NN155A. Pistils: Quantity: One per flower. Pistil length: About 2.5 cm. Style length: About 2.2 cm. Style color: Close to 84C to 84D. Stigma shape: Two-parted and curved. Stigma color: Close to N82A. Ovary color: Close to 145B. Seeds and fruits: Seed and fruit development have not been observed on plants of the new Hesperozygis.
Garden performance: Plants of the new Hesperozygis have been observed to have good garden performance and tolerate wind, rain and temperatures ranging from about 1° C. to about 40° C.
Pathogen & pest resistance: Plants of the new Hesperozygis have not been observed to be resistant to pathogens and pests common to Hesperozygis plants.

It is claimed:

1. A new and distinct Hesperozygis plant named ‘HES040302’ as illustrated and described.
